(19) United States
(12) Patent Application Publication
(10) Pub. No.: US 2020/0165177 A1
(43) Pub. Date: May 28, 2020

(54) Process and facility for producing propylene by combining propane hydrogenation and a steam cracking method with pre-separation steps in the two methods for partially removing hydrogen and methane

(71) Applicant: LINDE AKTIENGESELLSCHAFT, München (DE)

(72) Inventors: Torben HÖFEL, München (DE); Christine TÖGEL, München (DE); Mathieu ZELLHUBER, Planegg (DE); Heinrich LAIB, Ludwigshafen (DE); Stefan KOTREL, Ludwigshafen (DE); Martin DIETERLE, Ludwigshafen (DE); Florina Corina PATCAS, Ludwigshafen (DE); Sonja GIESA, Ludwigshafen (DE)

(21) Appl. No.: 16/630,323

(22) PCT Filed: Jul. 12, 2018
(86) PCT No.: PCT/EP2018/068996

§ 371 Date: Jan. 10, 2020

(30) Foreign Application Priority Data
Jul. 12, 2017 (EP) 17180974.2

(51) Int. Cl. C07C 5/327; C07C 7/00; C07C 7/04; C07C 7/09

(52) U.S. Cl. CPC C07C 5/327; C07C 7/005; C07C 11/06; C07C 7/09; C07C 7/04

(57) ABSTRACT

The invention concerns a process (10) for the production of propylene, which comprises carrying out a process (1) for propane dehydrogenation to obtain a first component mixture (A), carrying out a steam cracking process (2) to obtain a second component mixture (B), forming a first separation product (P1) which contains at least predominantly propylene using one or more first separation steps (S1), forming a second separation product (P2) containing at least predominantly propane using the first separation step(s) (S1), forming a third separation product (P3) containing at least predominantly ethylene using one or more second separation steps (S2) and forming a fourth separation product (P4) containing at least predominantly ethane using the second separation step(s) (S1). It is provided that at least a part of the first component mixture (A) is subjected to one or more first pre-separation steps (V1) to obtain a third component mixture (C), which pre-separation step(s) comprise a pressure increase and an at least partial removal of hydrogen, that at least a part of the second component mixture (B) is subjected to one or more second pre-separation steps (V2) to obtain a fourth component mixture (D), which comprises a pressure increase, an at least partial removal of hydrogen and an at least partial removal of methane, and in that at least part of the third component mixture (C) is subjected together with at least part of the fourth component mixture (D) to the first separation step or steps (S1). A corresponding plant and a process for converting a steam cracking plant are also the subject of the invention.

The present invention relates to a process and a plant for the production of propylene and to a process for retrofitting a steam cracking plant according to the preambles of the independent claims.

### PRIOR ART

Propylene (propene) is traditionally produced mainly by steam cracking of hydrocarbon feedstocks and other conversion processes in refinery processes. In these cases propylene is a minor by-product. Due to the increasing demand for propylene, especially polypropylene, propane dehydrogenation is also used.

Propane dehydrogenation is a well-known process in the petrochemical industry and is described in the article “Propene” in Ullmann's Encyclopedia of Industrial Chemistry, Online Edition 16 Sep. 2013, DOI: 10.1002/14356007.a22_211.pub3, in particular Section 3.3.1, “Propane dehydrogenation”.

Propane dehydrogenation is an endothermic equilibrium reaction generally carried out on noble or heavy metal catalysts, such as platinum or chromium. The dehydrogenation reaction is highly selective. For commercially available processes, total yields of approx. 90% are cited. Notwithstanding this high selectivity, smaller quantities of hydrocarbons with one, two, four and more than four carbon atoms are typically produced as by-products in addition to the hydrogen which is cleaved off. These by-products must be separated from the target product propylene.

Steam cracking processes and refinery processes in which propylene is formed are also described in literature, for example in the article “Ethylene” in Ullmann's Encyclopedia of Industrial Chemistry, online publication 15 Apr. 2009, DOI: 10.1002/14356007.a10_045.pub3, and in the article “Oil Refining” in Ullmann's Encyclopedia of Industrial Chemistry, online publication 15 Jan. 2007, DOI: 10.1002/14356007.a18_051.pub2.

In principle, the purification of a component mixture produced in propane dehydrogenation can at least partly be carried out together with the purification of a component mixture containing propylene from another process in which propylene is formed, e.g. a steam cracking process or a refinery process.

The combination of the purification of a component mixture from a propane dehydrogenation with the purification of a component mixture from a steam cracking process is known, for example, from U.S. Pat. No. 4,458,096 A or, specifically related to a cracking of hydrocarbons with two carbon atoms, from WO 2015/128039 A1, and a corresponding combination with the purification of a component mixture from a fluid catalytic cracking process, for example from U.S. Pat. No. 8,563,793 A or US 2010/331589 A1. However, these publications do not contain more detailed information regarding a corresponding combination. It should also be noted at this point that a fluid catalytic cracking process provides product mixtures with a fundamentally different composition than a steam cracking process, so that a combined separation must be designed differently here.

This invention aims to improve and make more efficient processes for the production of propylene, in which a component mixture from a propane dehydrogenation process and a component mixture from a steam cracking process are purified together.

### DISCLOSURE OF THE INVENTION

Against this background, the present invention proposes a process and plant for the production of propylene and a process for retrofitting a plant to perform a steam cracking process with the respective characteristics of the independent claims. Preferred embodiments are the subject of the dependent claims as well as the following description.

For a combined purification of components from different processes it is particularly advantageous if the respective component mixtures contain identical or similar components, i.e. the component mixtures do not “contaminate” each other with certain components not contained in the respective other component mixture (e.g. with hydrogen, carbon dioxide or oxygenates).

A combination of purification is particularly advantageous if the respective component mixtures have a similar concentration range, so that a synergetic separation process can be expected. However, this is normally not the case in practice. Furthermore, a combination of the purification is advantageous if one of the processes supplies significantly smaller quantities of a corresponding component mixture or if a corresponding plant is smaller and therefore separate purification is not worthwhile. This can be the case in particular if the steam cracking process has already been implemented in the form of a plant and a propane dehydrogenation system with significantly lower capacity is retrofitted. This could be particularly advantageous if some parts of the plant for carrying out the steam cracking process no longer run at full capacity due to a later change in use and these capacities can be used accordingly by the propane dehydrogenation process.

An essential aspect of the present invention is to pretreat a component mixture which is obtained using a propane dehydrogenation process, hereinafter also referred to as the “first” component mixture, in such a way that it is present in a state depleted of (at least) hydrogen and in particular at an increased pressure. The first component mixture is subjected to one or more pre-separation steps, which are subsequently referred to as the “first” pre-separation steps. The component mixture pre-treated in this way, which is subsequently referred to as the “third” component mixture, contains mainly hydrocarbons with three carbon atoms due to its pre-treatment. It may also contain smaller quantities of methane, residual hydrogen and hydrocarbons containing two carbon atoms and hydrocarbons containing four carbon atoms and, where appropriate, more than four carbon atoms.

A further essential aspect of the present invention is also to pretreat a component mixture obtained by using a steam cracking process, hereinafter also referred to as a “second” component mixture, in such a way that it is present in a (at least) hydrogen- and methane-depleted state and in particular also at an increased pressure. Here, the second component mixture is subjected to one or more pre-separation steps, which are subsequently also referred to as “second pre-separation steps”. The component mixture pretreated in this way, which is hereinafter referred to as the “fourth” component mixture if specifically referred to here, advantageously contains, due to its pre-treatment, predominantly hydrocarbons similar to those contained in the third component mixture and in a comparable concentration range, as well as comparable quantities of residual hydrogen and residual methane, unless completely separated. However, the second and fourth component mixtures may also contain significant amounts of hydrocarbons with four carbon atoms. This is the case, for example, when the fourth component mixture is present in the bottom of a deethanization column, as explained in detail below.

Since larger quantities of hydrocarbons with two carbon atoms, in particular ethane and ethylene, can generally be formed in a steam cracking process, in particular when lighter steam cracking inserts are used, a depletion of hydrocarbons with two carbon atoms can also take place in the course of the second pre-separation step(s) in the course of the depletion of hydrogen and methane. In other words, a demethanizer-first process or a deethanizer-first process can be used in the course of the second pre-separation step(s). The use of a depropanizer-first process is also possible in principle. Further details are explained below. However, even in the course of the first pre-separation step(s), a depletion of hydrocarbons with two carbon atoms can take place if this is appropriate.

The composition and pressure of the third and fourth component mixtures, which have been at least partially adjusted to each other by the first pre-separation step(s) and by the second pre-separation step(s), can be combined in a particularly advantageous manner and subsequently subjected to subsequent common separation steps. This makes it possible to design corresponding plant components for both processes together and thus to build a corresponding plant with lower investment costs and/or to operate it with lower operating costs.

Is there talk here of a component mixture being “depleted”, as compared to another component mixture, here especially the third compared as compared to the first and the fourth as compared to the second, in one or more components, here especially in hydrogen or hydrogen and methane, “depleted” is understood to mean that the depleted component mixture contains at most 0.5 times, 0.2 times, 0.1 times, 0.01 times or 0.001 times the content of the one or more components relative to the nondepleted component mixture and on a molar, mass or volume basis. Also a complete removal, i.e. a “depletion to zero” is understood here as a depletion. In the following, the term “predominantly” refers to a content of at least 60%, 80%, 90%, 95% or 99% on a molar, mass or volume basis.

Altogether, the present invention proposes a process for the production of propylene, which involves carrying out a propane dehydrogenation process to obtain a first component mixture containing at least hydrogen, ethane, ethylene, propane and propylene, and carrying out a steam cracking process to obtain a second component mixture containing at least hydrogen, methane, ethane, ethylene, propane and propylene. For the details of the respective processes and the product compositions typically formed, in particular also with regard to compounds contained in addition to the mentioned components, reference is made to the technical literature cited several times before. The propane dehydrogenation process is advantageously supplied with feeds containing propane and the steam cracking process with feeds rich in hydrocarbons. The latter are, for example, naphtha, but may also be lighter or heavier feeds, i.e. those which contain hydrocarbons with a higher and/or lower boiling point than are typically present in naphtha.

As is customary in this respect also in combined processes, a first separation product containing at least predominantly propylene is formed using at least part of the propylene of the first and second component mixtures and using one or more first separation steps and a second separation product containing at least predominantly propane is formed using at least part of the propane of the first and second component mixtures and using the first separation step or steps. The first separation step(s) may include in particular the use of a so-called C3 splitter, from which the first separation product can be taken at the top and the second separation product at the bottom. A C3 splitter of this type is typically preceded by further separation steps explained below.

In particular, the feed of a corresponding C3 splitter can typically be taken from a so-called depropanizer or a corresponding depropanizer column, as is also generally known from the cited technical literature. A depropanizer is a rectification column from which a gaseous fraction containing predominantly or exclusively hydrocarbons with three carbon atoms can be withdrawn at the top and a liquid fraction containing predominantly or exclusively hydrocarbons with four and optionally more carbon atoms can be withdrawn at the bottom. From other apparatuses associated with a corresponding rectification column which are part of a corresponding depropanizer, such as absorbers, corresponding fractions can also be removed instead of the rectification column itself or in addition to it. The fraction taken from the top of the depropanizer or corresponding apparatus can be fed to the C3 splitter. A corresponding depropanizer may be arranged at different locations in a separation sequence for processing a component mixture, in particular a component mixture obtained by a steam cracking process. In particular, a corresponding depropanizer can be arranged downstream of a deethanizer or a corresponding deethanizer column within the scope of the present invention and set up for the separation processing of a bottom product of the deethanizer. As explained below, a deethanizer can be used in the course of the present invention in the course of the second pre-separation step(s) or already in the course of the first separation step(s), depending on whether a deethanizer-first process or a demethanizer-first process is used. The same applies to a depropanizer, which can also be at the top of a corresponding separation sequence.

The present invention further comprises forming a third separation product containing at least predominantly ethylene using at least part of the ethylene of the first and second component mixtures and using one or more second separation steps, and forming a fourth separation product containing at least predominantly ethane using at least part of the ethane of the first and second component mixtures and using the second separation step or steps. The second separation step or steps typically involve the use of a so-called C2 splitter, from which the third separation product can be taken at the top and the fourth separation product at the bottom. A corresponding C2 splitter can in particular be fed with a fraction containing predominantly or exclusively ethane and ethylene which can be withdrawn from the top of a deethanizer, i.e. a corresponding rectification column or an apparatus associated therewith, in a demethanizer-first process and from the bottom of a demethanizer in a deethanizer-first process. All process variants are explained in more detail below with reference to the drawings and can be used within the scope of this invention.

As mentioned above, the present invention comprises pre-separation steps, wherein it is provided that at least part of the first component mixture is subjected to one or more first pre-separation steps to obtain a third component mixture, which comprises a pressure increase and at least partial removal of hydrogen, and that at least part of the second component mixture is subjected to one or more second pre-separation steps to obtain a fourth component mixture, which comprises a pressure increase, at least partial removal of hydrogen and at least partial removal of methane. It should already be noted at this point that, as mentioned above, at least partial removal of hydrocarbons with two carbon atoms can also be carried out in the context of the second pre-separation step(s). In the latter case, the present invention is used in connection with a deethanizer-first method, otherwise in connection with a demethanizer-first method. As mentioned above, this invention can also be used in conjunction with a depropanizer-first method.

Furthermore, the present invention provides that at least a part of the third component mixture together with at least a part of the fourth component mixture is subjected to the first separation step or steps. The present invention, with its respective embodiments explained below, provides different possibilities for combining the third component mixture with the fourth component mixture or the respective proportions used. In all cases, the main advantage of the present invention is that a particularly simple and efficient joint separation is possible in at least some of the components due to a comparable composition of the third and fourth component mixtures.

As already mentioned, this invention can be used in conjunction with a deethanizer-first process. In this case, when hydrogen and methane are removed in the second pre-separation step(s), ethane and ethylene are also at least predominantly removed, so that these do not predominantly transition from the second to the fourth component mixture or corresponding proportions. It should be noted that in such a deethanizer-first process, the fourth component mixture can also represent a bottom product of a deethanizer that is present in the bottom of a corresponding rectification column.

In this context, a “rectification column” is a separation unit which is designed to at least partially separate one or more gaseous or liquid component mixtures or in the form of a two-phase mixture with liquid and gaseous components, possibly also in the supercritical state, by rectification, i.e. to produce pure substances or at least substance mixtures with a different composition from the component mixture(s). Rectification is known to involve repeated evaporation and condensation processes, especially on or using suitable internals such as separating trays or structured or non-structured packings. A rectification column for use within the scope of this invention has a bottom evaporator. This is a device with a heat exchanger that is heated and is designed to heat a liquid fraction, also known as a bottom liquid, that accumulates in the bottom of the rectification column. Using a bottom evaporator, part of the bottom product is continuously evaporated and fed back into the rectification column in gaseous form. A rectification column for use in the context of the present invention also contains a top condenser which condenses gas rising in the rectification column and returns it to the rectification column in a condensed state.

For the design and specific configuration of rectification columns and other separation equipment, reference is made to relevant textbooks (see, for example, K. Sattler, “Thermische Trennverfahren: Grundlagen, Auslegung, Apparate”, 3rd edition, Wiley-VCH, Weinheim 2001).

As regards separation processes specifically used for the treatment of component mixtures formed by steam cracking, in particular separation processes involving demethanization and deethanization, reference is made to the already cited article “Ethylene” in Ullmann's Encyclopedia of Industrial Chemistry. Such separation processes differ in particular in the sequence of the respective separation steps. For example, the demethanizer-first process (also known as the front-end demethanizer process) and the deethanizer-first process (also known as the front-end deethanizer process), as well as the depropanizer first process (also known as the front-end depropanizer process) are known. As explained in detail below, this invention is particularly suitable for use in conjunction with the deethanizer-first method, but also for use with the demethanizer-first method or the depropanizer first method.

In particular, demethanizers, deethanizers and depropanizers, as mentioned above, may be designed as corresponding rectification columns or may include such rectification columns, which are hereinafter also referred to as demethanization columns, deethanization columns or depopanization columns. In the language used here, “demethanizers”, “deethanizers” and “depropanizers” are understood to mean arrangements with corresponding rectification columns, to which, however, additional apparatuses, such as absorbers in deethanizers, can also be assigned. The same applies if there is talk of “demethanization”, “deethanization” or “depropanization”. If it is mentioned below that fractions “from the top” or “from the bottom” can be withdrawn from demethanizers, deethanizers and depropanizers or corresponding rectification columns, these can also be withdrawn alternatively or in addition to the rectification column from the top or from the bottom of corresponding assigned apparatus.

According to an first preferred design of the present invention, the removal of hydrogen and methane in the second pre-separation step(s) also removes ethane and ethylene at least predominantly, i.e. a deethanizer-first process is carried out.

Hydrogen and methane are removed in the second pre-separation step(s), in which ethane and ethylene are also at least predominantly removed, using a deethanization column. In this case, the fourth component mixture is produced on a separating tray near the tray of such a deethanization column, whereby a “separating tray near the bottom” is understood as a separating tray which is arranged in the lower half, in particular in the lower third, in the lower quarter or in the lower fifth of the deethanization column. Compared to the second component mixture, a corresponding liquid is depleted of hydrogen, methane and hydrocarbons with two carbon atoms or corresponding components have been removed to a large extent.

It is particularly advantageous in such an embodiment of the present invention to feed the third component mixture at least partially in the liquid state in the area of a corresponding separating tray near the bottom, for example on such a separating tray or above thereof, into the deethanization column. This makes it possible to design the first preliminary separation step(s) in such a way that the third component mixture may contain residual amounts of hydrogen and, if appropriate, small amounts of methane and hydrocarbons with two carbon atoms, because these can still be removed in the deethanization column by means of the separation efficiency still present in the region of the separation tray near the bottom. This makes it possible to simplify the design of the first pre-separation step(s) because they do not have to be designed for the complete removal of hydrogen, methane and hydrocarbons with two carbon atoms.

Typically, in the context of this invention, a bottom liquid is drawn off from the bottom of the deethanization column and at least partially transferred to a depropanization column. This bottom liquid typically contains predominantly or exclusively hydrocarbons with three or more carbon atoms. In the depropanization column or an apparatus assigned to it, a top fraction is formed which predominantly or exclusively contains hydrocarbons with three carbon atoms. A bottom liquid of the depropanization column, on the other hand, exhibits predominantly or exclusively lower boiling hydrocarbons.

The third component mixture, in an embodiment of the present invention, can be fed at least partially, alternatively or in addition to the feed into the area of the separating tray near the bottom of the deethanization column, in the liquid state into the lower part, in particular also into the depropanization column. In such a case, it is typically not possible to remove lighter components such as residual hydrogen from the third component mixture in a targeted manner, but in such a case the hydrogen introduced can be used in a particularly advantageous way in a downstream hydrogenation process which is subjected to a fraction removed from the top of the depropanization column or an apparatus assigned to it, which contains predominantly or exclusively hydrocarbons with three carbon atoms. This makes it possible to dispense, at least in part, with a separate feed-in of additional hydrogen. As explained below, introduced hydrogen and other lighter compounds, in particular hydrocarbons with two carbon atoms, can be discharged in a stripper downstream of a corresponding hydrogenation.

The fraction drawn off from the top of the depropanization column or an apparatus associated therewith, which fraction contains predominantly or exclusively hydrocarbons having three carbon atoms and which fraction is optionally subsequently hydrogenated, is fed to a C3 splitter, in particular in the course of the first separation step or steps mentioned, in order to obtain the first and second separation product.

As mentioned several times, as an alternative to the first preferred embodiment of the invention described above, a second advantageous embodiment is also possible in which ethane and ethylene are at least predominantly not removed during the removal of hydrogen and methane in the second pre-separation step(s). Ethane and ethylene are therefore at least predominantly transferred to the fourth component mixture in these versions of the invention. In particular a demethanizer-first procedure is used. If a corresponding rectification column is used to remove hydrogen and methane, the fourth component mixture is in particular a bottom liquid of such a rectification column.

The removal of hydrogen and methane in the second pre-separation step(s), in which ethane and ethylene are at least predominantly not removed, can therefore be carried out using a demethanization column. A component mixture predominantly or exclusively containing hydrogen and methane is taken from the top of such a demethanization column or an apparatus associated with it, and a bottom liquid predominantly or exclusively containing hydrocarbons with two or more carbon atoms can be withdrawn from the bottom. This bottom liquid can in particular subsequently be deethanised, as explained below.

In the form of the present invention described above, the third component mixture can be fed into the demethanization column, in particular at least partially in the liquid state into a lower part, in particular an area of a separating floor near the bottom in the sense explained above, of the demethanization column. In principle, this results in the same advantage as explained previously with reference to the deethanizer-first process, namely that residual hydrogen and methane can still be discharged in the demethanization column. This also makes it possible in this process variant to design the first preliminary separation step(s) in such a way that residual hydrogen contents may be present in the third component mixture because these can still be removed in the demethanization column. This makes it possible to simplify the design of the first pre-separation step(s) because these do not have to be designed for complete removal of hydrogen and can therefore be designed more simply.

As mentioned above, a bottom liquid can be removed from the bottom of the demethanization column and at least partially transferred to a deethanization column. A component mixture containing predominantly or exclusively hydrocarbons with two carbon atoms, which is subsequently typically converted into a C2 splitter, i.e. subjected to the second separation step or steps, can be removed from such a deethanization column in a demethanizer-first process or from an apparatus associated therewith at the top. In addition, the deethanization column may be removed by a demethanizer-first process from a bottom liquid containing predominantly or exclusively hydrocarbons with three or more carbon atoms. In accordance with an embodiment of the present invention, it may also be provided to feed the third component mixture at least partially into an area of a separating tray near the bottom of this deethanization column.

It is advantageous to withdraw a bottom liquid from the bottom of the deethanization column and at least partially transfer it to a depropanization column. For the operating mode and the characteristics of such a depropanization column and the advantages that can be achieved in each case, reference is made to the explanations above.

As mentioned above, the third component mixture can be fed at least partially into the depropanization column. This applies in the same way to a deethanizer-first procedure and a demethanizer-first procedure. In both cases, a fraction containing propane and propylene may be withdrawn from the top of the depropanization column or an associated apparatus and at least partially hydrogenated with the addition of hydrogen and a hydrogenated fraction obtained.

As also mentioned above, after hydrogenation, components boiling higher than propane and propylene can be at least partially expelled from the hydrogenated fraction more easily. In this way, it is possible to remove components fed in as the third component mixture. For this no disadvantageous or disproportionate additional expenditure is necessary, since a corresponding expulsion of hydrogen is carried out anyway downstream of a typically occurring hydrogenation and therefore the other light components can also be expelled.

In a depropanizer-first process, with which the present invention can also be applied, the third component mixture can be fed into a rectification column downstream of this depropanization column.

In the context of this application, the term “stripping” or “expulsion” is understood to mean a separation step which involves the depletion of a component mixture of light components by increasing the temperature and/or passing a stripping or expulsion gas through it. For stripping, it is advisable to use a stripping column, or “stripper” for short, which has appropriately equipped means, e.g. a heating device and/or a feed facility for stripping gas. Likewise, a stripping column can have a capacitor and thus be very similarly constructed or equipped to a rectification column.

In particular, the hydrogen content of the first component mixture is depleted to a value of 0 to 10 mol %, in particular 0.1 to 5 mol %, for example 0.2 to 2 mol %, within the framework of the present invention in the context of the first pre-separation step or steps of the first component mixture. With such hydrogen contents, the second component mixture can be fed to the common separation, since its other composition is sufficiently similar to a corresponding fluid from a steam cracking process. As mentioned, any remaining hydrogen can simply be removed.

As mentioned above, the first pre-separation step or steps to which the first component mixture is subjected also include an increase in pressure, in particular to an absolute pressure of 3 to 40 bar, in particular 10 to 30 bar, for example 12 to 30 bar. The pressure level depends on a pressure level at which a demethanizer or deethanizer or depropanizer, as used in the second pre-separation step(s), is operated, i.e. the pressure increase also carried out there is carried out in the second pre-separation step(s). Therefore, the pressure levels of the third and fourth component mixtures can be adjusted in this way in a particularly advantageous way.

Hydrogen depletion as part of the pretreatment of the first component mixture or its part subjected to the pretreatment may include in particular a partial condensation of hydrocarbons with three carbon atoms after the pressure increase or compression described above. In this way, a fraction is formed which predominantly contains hydrocarbons with three carbon atoms, but into which the other components mentioned can also partially pass. In any case, this fraction is depleted of hydrogen compared to the first component mixture. Such condensation is particularly advantageous because, as explained below, it can be carried out at least in part within the context of the invention using refrigeration providable by process streams present in the process.

In particular, partial condensation may be carried out using refrigeration at least part of which may be obtained by decompressing a stream containing predominantly or exclusively propane. This stream, which mainly or exclusively contains propane, may, for example, be the second separation product formed in the first separation step(s). This second separation product can be expanded to produce cold and can then returned to the process, in particular the propane dehydrogenation process or the steam cracking process.

It is also possible to perform partial condensation using refrigeration generated at least in part by depressurising a part of the first component mixture or its portion subjected to the first pre-separation step(s). For example, the first component mixture or its portion subjected to the first pre-separation step(s) can be fed to the first pre-separation step(s) in the form of a stream of matter which is compressed and a partial stream of which is expanded downstream of compaction. The expanded partial stream can be fed back into the compression process, so that cold can be generated continuously.

So-called cold box processes, as they are generally known from the art, or processes based on other separation principles can also be used in the context of the invention.

It is particularly advantageous if the propane dehydrogenation process is carried out under water-free conditions and/or in the complete absence of oxygen (also in covalently bound form and/or during regeneration). In this way it becomes possible to form the first component mixture in such a way that neither water nor oxygen-containing compounds, especially carbon dioxide, are found in it. In this way, it is particularly easy to feed a corresponding first component mixture to the first pre-separation step(s) and, in particular, to the first separation step(s) because no separation of these components is required. In other words, the first component mixture can, without separating water and carbon dioxide during the formation of the third component mixture, be fed to a rectification column, for example, which serves to deethanize and which is typically operated at cryogenic temperatures at which water and carbon dioxide would freeze out.

As mentioned above, feeding the third component mixture after the first separation step(s) to the first separation step(s) in which the third component mixture is combined with the fourth component mixture or a component mixture formed therefrom is/are particularly advantageous if the respective compositions are identical or do not differ by more than a predetermined extent.

It is therefore particularly advantageous if a hydrogen content in the third component mixture differs from a hydrogen content in the fourth component mixture by not more than 50%, in particular by not more than 25%, for example by not more than 10%, of a hydrogen content in the third component mixture and if a content of hydrocarbons having three carbon atoms, in particular propylene, in the third component mixture differs from a content of hydrocarbons having three carbon atoms, in particular propylene, in the fourth component mixture by not more than 50%, in particular by not more than 25%, for example by not more than 10%.

The present invention further extends to a plant for the production of propylene, comprising a first reactor unit provided and arranged to perform a propane dehydrogenation process to obtain a first component mixture containing at least hydrogen, ethane, ethylene, propane and propylene, a second reactor unit arranged to perform a steam cracking process to obtain a second component mixture, containing at least hydrogen, methane, ethane, ethylene, propane and propylene, a first separation unit provided and arranged to form a first separation product containing at least predominantly propylene using at least a portion of the propylene of the first and second component mixtures and using one or more first separation steps, wherein the first separation unit is further provided and arranged to form a second separation product containing at least predominantly propane using at least a portion of the propane of the first and second component mixtures and using the first separation step or steps, and a second separation unit arranged to form a third separation product containing at least predominantly ethylene, is provided and arranged using at least a portion of the ethylene of the first and second component mixtures and using one or more second separation steps, the second separation unit further being provided and arranged to form a fourth separation product containing at least predominantly ethane using at least a portion of the ethane of the first and second component mixtures and using the one or more second separation steps.

According to the invention, such a plant is characterized by a first pre-separating unit provided and arranged to subject at least a part of the first component mixture to one or more first pre-separating steps while obtaining a third component mixture, which comprises a pressure increase and an at least partial removal of hydrogen, a second pre-separating unit provided and arranged for this purpose, subjecting at least a portion of the second component mixture to one or more second pre-separation steps to obtain a fourth component mixture comprising a pressure increase, an at least partial removal of hydrogen and an at least partial removal of methane, and means provided and arranged to feed at least a portion of the third component mixture together with at least a portion of the fourth component mixture to the first separation unit and subject the first separation step or steps to the fourth component mixture.

With regard to the features and advantages of the inventionally proposed plant, reference is made explicitly to the features and advantages already described with regard to the explained process and its advantageous embodiment in the explained features and advantages. The same applies in particular to a plant in accordance with a particularly preferred form of the present invention, which has means which have been set up to carry out a corresponding procedure.

The invention further comprises a process for retrofitting a plant adapted to perform a steam cracking process using a plurality of plant components such as cracking furnaces, processing equipment and separating apparatus, wherein a hydrocarbon-containing feed mixture having a first composition is fed to the plant prior to retrofitting. In accordance with the invention, the retrofitting comprises adding a hydrocarbon-containing feed mixture with a second, different composition to the plant instead of the hydrocarbon-containing feed mixture with the first composition, and using one or more of the plant components for a propane dehydrogenation process instead of for the steam cracking process, i.e. reallocating freed capacities accordingly.

An example of this is a change in the feedstock mix of the steam cracking process from heavier hydrocarbons, for example predominantly naphtha, to lighter hydrocarbons, for example ethane and/or propane and butane. While certain plant components for processing the entire product gas, such as the raw gas compressor, and plant components for processing the light product fraction, such as the demethanizer, are likely to be subjected to the same or even higher loads than before after the change in the feed mixture, other plant components, for example for processing heavier product fractions, are likely to be relieved. These relieved plant components can include the depropanizer as well as all plant components for processing a fraction of hydrocarbons with three carbon atoms including hydrogenation and a splinter. These plant components can then also be used for a propane dehydrogenation process. The procedure described in the present invention for removing hydrogen from the product gas of propane dehydrogenation is particularly advantageous here, since this procedure does not place an even greater load on the heavily utilised existing plant components of the steam cracking process, such as the demethanizer.

According to the invention, a corresponding modification includes carrying out a procedure as described and/or providing a corresponding installation. In this way, the advantage mentioned at the outset can be achieved that the corresponding products of a propane dehydrogenation can be purified together with the products of the steam cracking process and separate purification can be dispensed with.

The invention is explained in more detail below with reference to the attached drawings, in which a preferred form of execution of the present invention is explained compared to the state of the art.

### SHORT DESCRIPTION OF THE DRAWINGS

FIG. 1 illustrates a process designed according to an embodiment of the invention in a simplified, schematic representation.

FIG. 2 illustrates a process designed according to an embodiment of the invention in a simplified, schematic representation.

FIG. 3 illustrates process alternatives according to embodiments of the invention as well as process alternatives that are not part of the invention.

FIG. 4 continues the representation of FIG. 3.

In the figures, constructionally and/or functionally corresponding elements are indicated with identical reference signs and are not explained repeatedly for the sake of clarity.

### DETAILED DESCRIPTION OF THE DRAWINGS

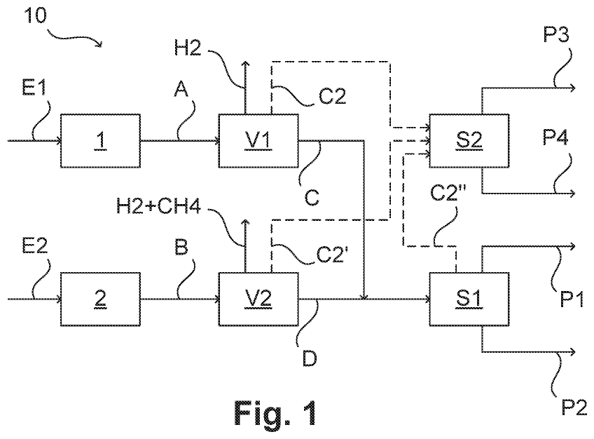

FIG. 1 illustrates a process designed according to the invention in a highly simplified, schematic representation and is designated 10 in total.

Process 10 comprises a process 1 for propane dehydrogenation, a steam cracking process 2, one or more first preliminary separation steps V1, one or more second preliminary separation steps V2, one or more first separation steps S1 and one or more second separation steps S2. The first pre-separation step(s) V1, the second pre-separation step(s) V2, the first separation step(s) S1 and the second separation step(s) S2 can each be grouped as required and, for example, combined in corresponding plant components. An essential aspect of process 10, however, is that the first pre-separation step(s) V1 and the second pre-separation step(s) V2 are each performed separately, i.e. component mixtures fed to the first pre-separation step(s) are not fed to the second pre-separation step(s) at least at the same location as other component mixtures and vice versa.

In the example shown, process 1 for propane dehydrogenation is supplied with an initial input stream E1, which may include propane in particular. Process 1 for propane dehydrogenation is carried out in a generally known manner so that a first component mixture A containing at least hydrogen, ethane, ethylene, propane and propylene is formed therein and can be withdrawn from process 1 for propane dehydrogenation in the form of a corresponding material stream. Procedure 1 for propane dehydrogenation may in particular be carried out using one or more suitable reactors, which may have been designed in a customary manner.

In the example shown, the first component mixture A or the corresponding material flow is at least partially fed to the first pre-separation step(s) V1 in which the first component mixture A or the corresponding material flow is subjected to a pressure increase and at least partial removal of hydrogen. As mentioned above, this can be done in a generally known way. In particular, the first component mixture A or the corresponding material flow can be liquefied in the first pre-separation step(s) V1. Separated hydrogen is illustrated in the form of a material stream designated H2. Also at least partial removal of hydrocarbons with two carbon atoms is possible, however optional, as shown by dotted line of material flow C2. In this way, a component mixture is obtained, which is also referred to here as the third component mixture C, and which can be withdrawn in the form of a corresponding material flow from the first pre-separation step(s) V1.

Possible hydrogen contents of the third component mixture C or the corresponding material flow have already been explained above. In particular, the third component mixture C downstream of the first preliminary separation step(s) V1 still contains hydrocarbons with three carbon atoms and minor amounts of other components, for example hydrocarbons with two hydrocarbon atoms, if not yet removed, and hydrocarbons with four carbon atoms formed as by-products in propane dehydrogenation process 1.

If the first component mixture A also contains other components such as water and carbon dioxide, these can also be removed in the first pre-separation step(s) V1.

The steam cracking process 2, which can also be carried out in the usual manner, for example by using several cracking furnaces, is fed a hydrocarbon-rich feed in the form of a material flow E2. The hydrocarbon-rich feed can include in particular naphtha and lighter hydrocarbons, but also heavy hydrocarbons. In particular, hydrocarbon-rich feeds may include paraffinic hydrocarbons with two, three and four carbon atoms, in particular ethane, propane and butane. The steam cracking process 2 as a whole or different furnaces used in the steam cracking process 2 can also be supplied with different hydrocarbon feedstocks and processed there under different cracking conditions. In the steam cracking process 2, the hydrocarbons of the hydrocarbon-rich feed(s) are at least partially converted so that a second component mixture B is obtained which contains at least hydrogen, methane, ethane, ethylene, propane and propylene. The second component mixture B can be drawn off from the steam cracking process 2 in the form of a corresponding material flow and then at least partly fed to one or more second pre-separation steps V2. The composition of the second component mixture B or of the corresponding material flow depends to a large extent on the hydrocarbon-rich application supplied to the steam cracking process 2.

As already explained several times, the second pre-separation step(s) V2 may include demethanization or deethanization in particular. In both cases the pressure is increased. Demethanization involves at least partial removal of hydrogen and at least partial removal of methane from the second component mixture B, as illustrated in the form of a material stream designated H2+CH4. Ethane and ethylene or generally hydrocarbons with two carbon atoms are not removed from the second component mixture B, however. In contrast, deethanization also involves at least partial removal of hydrogen and methane, so that in this case, too, the material flow designated H2+CH4 is formed, but also at least partial removal of hydrocarbons with two carbon atoms. The latter is illustrated by the dashed material flow C2′. In a deethanizer-first process, the H2+CH4 material stream leaves the top of a demethanization column downstream of the deethanization column or an apparatus associated with the demethanization column, and the C2′ material stream leaves the bottom of the demethanization column. In a demethanizer first process, in which no corresponding C2′ material stream is formed, the H2+CH4 material stream leaves the top of a demethanization column or an apparatus associated therewith, but in the bottom of the column there is a component mixture which, in addition to hydrocarbons with two carbon atoms, also contains heavy hydrocarbons, so that the C2′ material stream cannot be withdrawn from the bottom of the demethanization column. This is deducted from the top of a deethanization column downstream of the demethanization column or an apparatus assigned to a deethanization column.

Irrespective of the process specifically carried out, however, in both cases a component mixture D is formed by the use of the second pre-separation step(s), which component mixture D is referred to here as the fourth component mixture, and which is depleted with respect to the second component mixture B at least in hydrogen and in methane, or which is formed by at least partially removing hydrogen from methane from the second component mixture B. The second component mixture B is at least partially depleted with respect to the second component mixture B, and the second component mixture B is at least partially depleted with respect to hydrogen.

This fourth component mixture can be physically withdrawn in the form of a corresponding material flow from the second pre-separation step(s) V2. The present invention, however, also includes the fact that such a fourth component mixture D is present in a device used in the second pre-separation step V2. For example, the fourth component mixture D, as explained several times, can be a liquid in the area of a separating tray near the bottom of a demethanization or deethanization column, depending on whether a deethanizer-first process or a demethanizer-first process is used. This liquid is also depleted compared to the second component mixture B at the previously described components (hydrogen, methane and possibly hydrocarbons with two carbon atoms) or this was formed by at least partially removing the corresponding components from the second component mixture B. The liquid is then separated from the second component mixture B by the addition of a second component mixture B. The second component mixture B is then separated from the second component mixture B by the addition of a second component mixture B.

As also explained below, the already mentioned third component mixture C can be added to the area of the separation tray near the bottom of a corresponding demethanization or deethanization column, so that from the bottom of the demethanization or deethanization column not the fourth component mixture D but a further component mixture is subtracted, which results from the combination of the third component mixture C with the fourth component mixture D and the separation effect still present to a small extent in the region of the separating tray near the bottom of the demethanization or deethanization column. However, the third component mixture C can also be fed downstream of the demethanization or deethanization column instead of into the area of the separating tray near the bottom.

However, in all cases described, the first separation step(s) S1 is/are carried out in the course of process 10, in which two separation products are formed, namely a first separation product P1 and a second separation product P2. The first separation product P1 comprises predominantly or exclusively propylene, the second separation product P2 predominantly or exclusively propane. The second separation product P2 can in particular be returned to process 10. The first separation product, P1, is one of the products of process 10.

The propylene of the first separation product P1 and the propane of the second separation product P2 each originate at least partly from both the first component mixture A and the second component mixture B and thus also at least partly from both the third component mixture C and the fourth component mixture D. In other words, the present invention proposes an at least partially combined recovery of the first separation product P1 and the second separation product P2 from a first component mixture A originating from a propane dehydrogenation process 1 and from a second component mixture B originating from a steam cracking process 2 or from third and fourth component mixtures C and D formed therefrom.

The first separation steps S1 typically comprise the use of a C3 splitter in the final stage, as already explained several times, to which a mixture of predominantly or exclusively hydrocarbons with three carbon atoms, in particular propylene and propane, is added. A corresponding hydrogenation can be provided upstream of a corresponding C3 splitter, as also mentioned above. Again, upstream of this hydrogenation, which can optionally be omitted, a depropanization can be provided in particular, which can be carried out using a depropanization column. A component mixture which predominantly or exclusively contains hydrocarbons having three carbon atoms can be taken from the top of a corresponding depropanization column or an apparatus assigned to the depropanization column, and a component mixture with heavier hydrocarbons can be taken from the bottom. The component mixture taken from the top side of the depropanization column or the associated apparatus can be fed to the C3 splitter, if necessary after the optional hydrogenation mentioned above.

In turn, depropanization may be preceded by demethanization and deethanization (in the case of a demethanizer-first process) or deethanization (in the case of a deethanizer-first process) in the order indicated in each case, wherein the first separation step (demethanization in the demethanizer-first process or deethanization in the deethanizer-first process) in each case in accordance with these alternatives represents at least in part a part of the second preliminary separation step(s) V2 or one of the separation steps used here in the systematic used here.

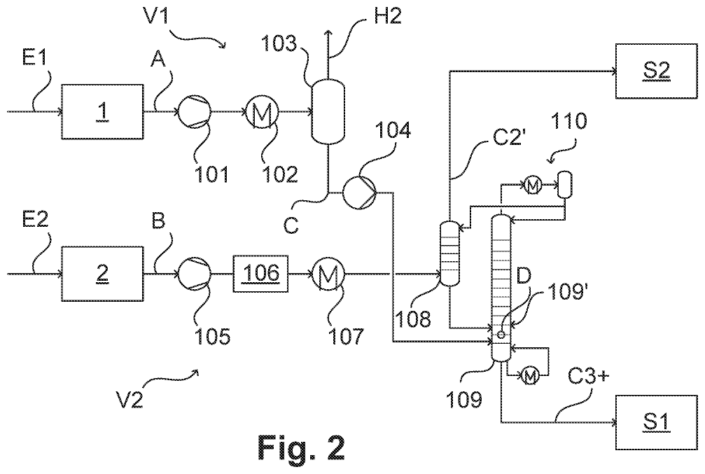

In both cases, a component mixture containing predominantly or exclusively hydrocarbons with three or more carbon atoms is taken from the bottom of a deethanization column used in deethanization. This is then fed into the depropanization process. Further details are given in FIG. 2 below. In a demethanizer first process, a bottom product of demethanization, which is only depleted of hydrogen and methane or freed of them, but still contains hydrocarbons with two or more carbon atoms, is partially or completely added to the deethanization. In a deethanizer-first process, a top product of deethanization containing hydrocarbons with two and fewer carbon atoms and hydrogen is partially or completely added to the demethanization, while a bottom product of deethanization containing hydrocarbons with three and more carbon atoms is partially or completely added to the depropanization.

The third component mixture C can in particular be fed to the area of a bottom near the bottom of a demethanization column, the area of a bottom near the bottom of a deethanization column or a depropanization column as explained in detail above. The “joint” separation proposed by the present invention begins at the respective entry point.

Process 10 further comprises one or more second separation steps S2 in which a third separation product P3 and a fourth separation product P4 are formed. The third separation product P3 consists predominantly or exclusively of ethylene, whereas the fourth separation product P4 consists predominantly or exclusively of ethane.

Both the ethylene of the third separation product P3 and the ethane of the fourth separation product P4 originate at least partly from both the first component mixture A and the second component mixture B and thus also at least partly from both the third component mixture C and the fourth component mixture D. In particular, the fourth separation product P4 can be recycled in process 10.

In particular, the second separation step or steps may include the use of a C2 splitter into which a mixture of predominantly or exclusively hydrocarbons containing two carbon atoms can be fed, in particular a mixture of ethylene and ethane. Such a mixture may be provided in particular in a deethanizer-first process from the bottom of a demethanization column and in a demethanizer-first process from the top of a deethanization column or an apparatus associated with the deethanization column. As mentioned above, demethanization or deethanization in the systematics used here can also be regarded at least in part as part of the second pre-separation step V2.

As already mentioned, in the first pre-separation step(s) V1 and in the second pre-separation step(s) V2 material flows C2 and C2′ can be separated, which predominantly or exclusively comprise hydrocarbons with two carbon atoms. However, this is possible alternatively or additionally only in the first separation step(s). The latter is the case, for example, if a demethanizer-first process is used in the second separation step(s) V2. In this case, too, the third component mixture C can be fed into the area of a bottom near the bottom of a demethanization column. The third component mixture C and the fourth component mixture D are therefore already combined at this point. In this case, the subsequent deethanization can be systematically regarded as part of the first separation step(s) so that, as shown here in the form of a dashed C2″ stream, a stream containing predominantly or exclusively hydrocarbons with two carbon atoms is only formed there. The material flows C2′ and C2″ are thus formed advantageously as alternatives to each other, the material flow C2 can be formed in addition.

FIG. 2 illustrates a process designed in accordance with the invention in a simplified schematic representation, which, however, shows further details of a specific embodiment compared to the representation in FIG. 1. Elements that are structurally identical or comparable or functionally identical or comparable are shown with identical reference signs as in FIG. 1 and are not explained repeatedly for the sake of clarity.

As shown in FIG. 2, in the first pre-separation step(s) V1, the first component mixture A is first compressed in a compressor 101 and then cooled in a heat exchanger 102. At least some of the hydrocarbons with three and optionally two carbon atoms contained in the first component mixture A are condensed out.

For phase separation, the correspondingly compressed and cooled first component mixture A can be transferred to a phase separator 103. From the top of the phase separator 103, the material flow already shown in FIG. 1, marked H2, can be subtracted. The third component mixture C is drawn off from the bottom of the phase separator 103 and can be conveyed further by means of a pump 104.

The second pre-separation step(s) V2 also includes a first compression of the second component mixture B using a compressor 105, followed by a drying in a dryer 106. In addition, carbon dioxide can also be removed before or between compaction or drying (not shown in the picture). After drying or removal of carbon dioxide, the second component mixture B is cooled using a heat exchanger 107.

The second component mixture B, which has been dried accordingly and possibly freed of carbon dioxide, is now added to a deethanization process, so the embodiment illustrated in FIG. 2 uses a deethanizer-first process. Here the component mixture B is first fed into an absorber column 108, which is fed with a liquid return which is formed using a top gas from the actual deethanization column 109. A bottom product is fed to the deethanization column in absorber column 108. In the deethanization column 109 a liquid forms in the area of a bottomy ground 109′, which represents the fourth component mixture D in the systematics used here. The third component mixture C is fed in at this point. A top gas can be drawn off from the top of the deethanization column 109, which is operated with a bottom evaporator, liquefied in a top condenser marked 110 in total and led back to the absorber column 108 in a first part and to the deethanization column 109 in another part.

From the bottom of the deethanization column 109, a material stream containing predominantly or exclusively hydrocarbons with three or more carbon atoms can be withdrawn, which is designated here as C3+, from the top of the absorber column 108 and thus as a gaseous fraction formed during deethanization, the material stream C2′ already illustrated in FIG. 1 can be withdrawn. The former may be added to the first separation step(s) S1, the latter to the second separation step(s) S2, the respective separation products P1 to P4 of which are not separately illustrated in FIG. 2.

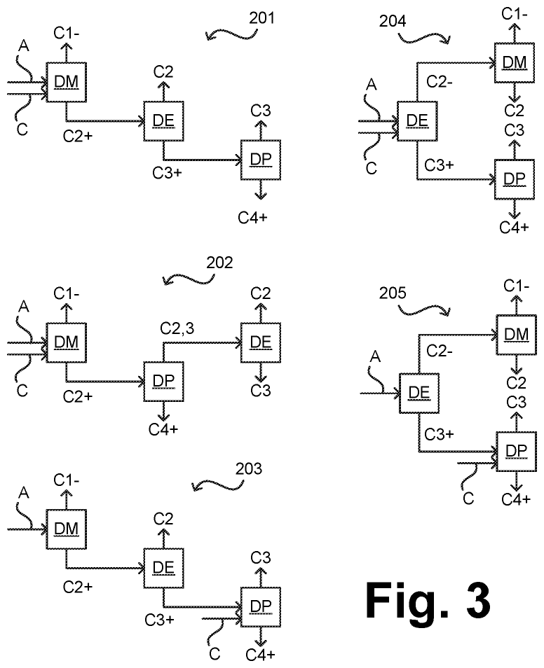

FIG. 3 illustrates the different forms of inventive methods in a very simplified way. The different feeding options for the third component mixture into either demethanization (DM), deethanization (DE) and depropanization (DP) are illustrated here. Component mixtures containing predominantly or exclusively hydrogen and methane are indicated with C1−, component mixtures containing predominantly or exclusively hydrocarbons with two carbon atoms and lighter components with C2−, component mixtures containing predominantly or exclusively hydrocarbons with two carbon atoms with C2, component mixtures containing predominantly or exclusively hydrocarbons with three carbon atoms and lighter components with C3−, component mixtures containing predominantly or exclusively hydrocarbons with three carbon atoms with C3, component mixtures containing predominantly or exclusively hydrocarbons with two or more carbon atoms with C2+, component mixtures containing predominantly or exclusively hydrocarbons with three or more carbon atoms with C3+, component mixtures containing predominantly or exclusively hydrocarbons with four or more carbon atoms with C4+, and component mixtures predominantly or exclusively containing hydrocarbons with two or three carbon atoms with C2,3. The component mixtures already indicated A to D in FIGS. 1 and 2 are also illustrated here and indicated accordingly.

According to alternative 201, a demethanizer-first process is used. The third component mixture C is fed into the area of a separating tray near the bottom of a demethanization column used in demethanization DM. The other steps of deethanization DE and depropanization DP follow in a basically well-known way.

According to alternative 202, a demethanizer-first process is also used. The third component mixture is also fed into the area of a separating tray near the bottom of a demethanization column used in demethanization DM. In contrast to alternative 201, however, here the depropanization DP is followed by deethanization DE, in a per se well-known manner.

Alternative 203 also uses a demethanizer-first process, wherein the sequence is basically the same as in alternative 201, but the third component mixture C of a depropanization column used in DP depropanization is fed in. Accordingly, the top product of this depropanization could now also contain small amounts of hydrogen, methane and hydrocarbons with two carbon atoms, provided these have not been completely removed from the component mixture C beforehand. These light components could then be removed by a stripper downstream of the subsequent hydrogenation. The top gas of this stripper, which now contains the ethylene as well as the ethane from the first component mixture A, could then be fed directly to the second separation step(s) S2 or indirectly to the second separation step(s) S2 via recompression in the first separation step(s) V1 or V2.

According to the alternatives 204 and 205, deethanizer-first processes are used in each case, wherein deethanization DE is followed by Demethanization DM and Depropanization DP in basically known ways. The alternatives 204 and 205 differ in the respective feed of the third component mixture C, which is fed into the area of a separating tray near the bottom of a demethanization column used in deethanization DE in alternative 204 and into a depropanization column used in depropanization DP in alternative 205. For alternative 205, the same applies for the overtop product of this depropanization as for alternative 203.

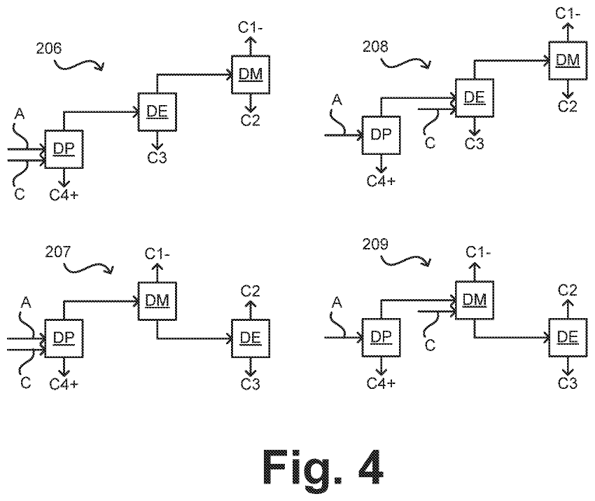

FIG. 4 continues the representation of FIG. 3, wherein depropanizer-first methods are illustrated here in the form of alternatives 206 to 209. In alternatives 206 and 207, which are not in line with the invention, the third component mixture C is fed into a depropanization column used in depropanization DP, in alternative 208 into the area of a separating tray near the bottom of a demethanization column used in deethanization DE and in alternative 209 into the area of a separating tray near the bottom of a demethanization column used in demethanization. The sequence of the remaining steps results directly from the drawing. For alternatives 208 and 209, the small quantities of hydrocarbons with four carbon atoms from the component mixture C finally reach the separation product P2, which mainly contains propane. This is not critical if the separation product P2 is used as a recycle for the steam cracking process.

Finally, as already mentioned, the component mixture C can also be fed independently of the separation sequences described above upstream of the processing of hydrocarbons with three carbon atoms, for example before the hydrogenation of corresponding hydrocarbons (not shown in the drawing). Accordingly, the hydrocarbons with two or fewer carbon atoms, as described for alternative 203 and 205, would still have to be added to the second separation step(s) S2. In addition, small amounts of hydrocarbons with four or more carbon atoms in the separation product P2 would be tolerable, as described for alternatives 208 and 209.

1. Process (10) for the production of propylene, which comprises:
performing a propane dehydrogenation process (1) to obtain a first component mixture (A) containing at least hydrogen, ethane, ethylene, propane and propylene,
performing a steam cracking process (2) to obtain a second component mixture (B) containing at least hydrogen, methane, ethane, ethylene, propane and propylene,
forming a first separation product (P1) containing at least predominantly propylene using at least part of the propylene of the first and second component mixtures (A, B) and using one or more first separation steps (S1),
forming a second separation product (P2) containing at least predominantly propane using at least a portion of the propane of the first and second component mixtures (A, B) and using the first separation step or steps (S1),
forming a third separation product (P3) containing at least predominantly ethylene using at least part of the ethylene of the first and second component mixtures (A, B) and using one or more second separation steps (S2),
forming a fourth separation product (P4) containing at least predominantly ethane using at least a portion of the ethane of the first and second component mixtures (A, B) and using the second separation step or steps (S1), and
characterized in
that at least part of the first component mixture (A) is subjected to one or more first pre-separation steps (V1), which comprise a pressure increase and an at least partial removal of hydrogen, to obtain a third component mixture (C),
that at least part of the second component mixture (B) is subjected to one or more second pre-separation steps (V2), which comprise a pressure increase, an at least partial removal of hydrogen and an at least partial removal of methane, to obtain a fourth component mixture (D), and
in that at least part of the third component mixture (C) together with at least part of the fourth component mixture (D) are subjected to the first separating step or steps (S1).

2. The process (10) according to claim 1, in which ethane and ethylene are also at least predominantly removed during the removal of hydrogen and methane in the second preliminary separation step or steps (V2), wherein the removal of hydrogen and methane in the second preliminary separation step or steps (V2), in which ethane and ethylene are also at least predominantly removed, is carried out using a deethanization column (DE).

3. The process (10) according to claim 2, in which the third component mixture is fed at least partially in the liquid state into the region of a separating tray near the bottom of the deethanization column (DE).

4. The process (10) according to claim 1, in which ethane and ethylene are at least predominantly not removed during the removal of hydrogen and methane in the second preliminary separation step(s) (V2), wherein the removal of hydrogen and methane in the second preliminary separation step(s) (V2), in which ethane and ethylene are at least predominantly not removed, is carried out using a demethanization column (DM).

5. The process (10) according to claim 4, in which the third component mixture is fed at least partially in the liquid state into the region of a separating tray near the bottom of the demethanization column (DM).

6. The process (10) according to claim 4 or 5, in which a bottom liquid is withdrawn from the bottom of the demethanization column (DM) and at least partially transferred into a deethanization column (DM).

7. The process (10) according to claim 2 in which a bottom liquid is drawn off from the bottom of the deethanization column (DE) and at least partially transferred to a depropanization column (DP).

8. The process (10) according to claim 7, in which the third component mixture is fed at least partially in the liquid state to the depropanization column (DP).

9. The process (10) according to claim 8, in which a fraction containing propane and propylene is drawn off from the top of the depropanization column (DP) or an apparatus associated with the depropanization column (DP) and subjected at least partially to hydrogenation with the addition of hydrogen and with a hydrogenated fraction being obtained, wherein after the hydrogenation components boiling more easily than propane and propylene are at least partially expelled from the hydrogenated fraction.

10. The process (10) according to claim 1, in which in the first preliminary separation step or steps (V1) of the first component mixture its hydrogen content is depleted to a value of 0 to 10 mol %, in particular 0.1 to 5 mol %, for example 0.2 to 2 mol %.

11. The process (10) according to claim 1, in which the first pre-separation step or steps to which the first component mixture is subjected comprise an increase in pressure to an absolute pressure of 3 to 40 bar, in particular of 10 to 30 bar, for example of 12 to 30 bar.

12. The process (10) according to claim 11 in which at least partial condensation of components boiling heavier than hydrogen is carried out in the first pre-separation step or steps after the pressure increase.

13. A plant for the production of propylene with:
a first reactor unit provided and arranged to perform a propane dehydrogenation process (1) to obtain a first component mixture (A) containing at least hydrogen, ethane, ethylene, propane and propylene,
a second reactor unit provided and arranged to perform a steam cracking process (2) to obtain a second component mixture (B) containing at least hydrogen, methane, ethane, ethylene, propane and propylene,
a first separation unit provided and arranged to form a first separation product (P1) containing at least predominantly propylene using at least a portion of the propylene of the first and second component mixtures (A, B) and using one or more first separation steps (S1),
wherein the first separation unit is further provided and arranged to form a second separation product (P2) containing at least predominantly propane using at least a portion of the propane of the first and second component mixtures (A, B) and using the first separation step or steps (S1),
a second separation unit provided and arranged to form a third separation product (P3) containing at least predominantly ethylene using at least a portion of the ethylene of the first and second component mixtures (A, B) and using one or more second separation steps (S2),
wherein the second separation unit is further provided and arranged to form a fourth separation product (P4) containing at least predominantly ethane using at least part of the ethane of the first and second component mixtures (A, B) and using the second separation step or steps (S1), and
characterized by
a first pre-separation unit provided and arranged to subject at least a portion of the first component mixture (A) to one or more first pre-separation steps (V1) comprising a pressure increase and an at least partial removal of hydrogen to obtain a third component mixture (C),
a second pre-separating unit provided and adapted to subject at least a portion of the second component mixture (B) to one or more second pre-separating steps (V2) comprising a pressure increase, at least partial removal of hydrogen and at least partial removal of methane to obtain a fourth component mixture (D), and
means provided and arranged to feed at least a part of the third component mixture (C) together with at least a part of the fourth component mixture (D) to the first separation unit and to subject it to the first separation step or steps (S1).

14. A method of retrofitting a plant adapted to perform a steam cracking process using a plurality of plant components, wherein a hydrocarbon-containing feed mixture having a first composition is supplied to the plant prior to retrofitting, characterised in that the retrofitting comprises supplying a hydrocarbon-containing feed mixture having a second, different composition to the plant in place of the hydrocarbon-containing feed mixture having the first composition, and using one or more of the plant components in place of the steam cracking process for a propane dehydrogenation process, wherein a process according to claim 1 is carried out.

15. The process (10) according to claim 5, in which a bottom liquid is withdrawn from the bottom of the demethanization column (DM) and at least partially transferred into a deethanization column (DM).

16. The process (10) according to claim 3 in which a bottom liquid is drawn off from the bottom of the deethanization column (DE) and at least partially transferred to a depropanization column (DP).

17. The process (10) according to claim 2, in which in the first preliminary separation step or steps (V1) of the first component mixture its hydrogen content is depleted to a value of 0 to 10 mol %, in particular 0.1 to 5 mol %, for example 0.2 to 2 mol %.

18. The process (10) according to claim 3, in which in the first preliminary separation step or steps (V1) of the first component mixture its hydrogen content is depleted to a value of 0 to 10 mol %, in particular 0.1 to 5 mol %, for example 0.2 to 2 mol %.

19. The process (10) according to claim 4, in which in the first preliminary separation step or steps (V1) of the first component mixture its hydrogen content is depleted to a value of 0 to 10 mol %, in particular 0.1 to 5 mol %, for example 0.2 to 2 mol %.

20. A method of retrofitting a plant adapted to perform a steam cracking process using a plurality of plant components, wherein a hydrocarbon-containing feed mixture having a first composition is supplied to the plant prior to retrofitting, characterised in that the retrofitting comprises supplying a hydrocarbon-containing feed mixture having a second, different composition to the plant in place of the hydrocarbon-containing feed mixture having the first composition, and using one or more of the plant components in place of the steam cracking process for a propane dehydrogenation process, wherein a plant according to claim 13 is provided.
